(19) United States
(12) Patent Application Publication
(10) Pub. No.: US 2015/0139515 A1
(43) Pub. Date: May 21, 2015

(54) MOVEMENT CORRECTION FOR MEDICAL IMAGING

(71) Applicant: The State Of Queensland Acting Through Its Department of Health, Brisbane, Queensland, OT (AU)

(72) Inventors: Jye Smith, Drewvale (AU); Paul Thomas, Moorooka (AU)

(73) Assignee: The State of Queensland Acting Through Its Department of Health, Brisbane, Queensland (AU)

(21) Appl. No.: 14/411,351

(22) PCT Filed: Jul. 3, 2013
(86) PCT No.: PCT/AU2013/000724

§ 371 Date: Dec. 24, 2014

(30) Foreign Application Priority Data
Jul. 3, 2012 (AU) 2012902831

(51) Int. Cl. G06T 11/00; A61B 5/055; A61B 6/03; A61B 5/00

(52) U.S. Cl. CPC G06T 11/005; A61B 5/721; A61B 5/055; A61B 6/032; A61B 6/037; G06T 2211/412; G06T 2207/10072; G06T 2207/30004; G06T 2207/30196; G06T 2211/40; G06T 2211/416
USPC 382/131

(57) ABSTRACT

A method of improving the resolution of images from medical imaging devices by removing blurring due to movement of a patient during a scan. The method uses tracking algorithms to extract movement data from a video image of the patient and uses the movement data to correct the scanner date and remove the effects of movement. Also disclosed is a calibration process to calibrate the movement data to the scanner data.

### FIELD OF THE INVENTION

The present invention relates to the field of medicine and movement tracking. More particularly, the invention relates to correcting scan data to correct for patient movement, in particular head movement.

### BACKGROUND TO THE INVENTION

There have been numerous medical scanning techniques developed over recent years. Some of these techniques have relatively long data acquisition time, during which the patient should remain as still as possible. Any movement of the patient during a scan results in lower image quality. This can be a significant problem for scanning modalities such as computed tomography (CT), magnetic resonance imaging (MRI) and positron emission tomography (PET). For these modalities the limitation on image quality is often not intrinsic to the technique or equipment, but rather patient movement. A PET scan could achieve resolution of better than 2 or 3 mm, but for the patient movement that occurs over the course of the scan.

A typical PET scan of the head may take from 5 to 15 minutes, and some research scans 75 minutes or more. It is very difficult for a patient to hold their head completely stationary for this period of time. It is not unusual for a patient to fall asleep, which can result in movement of the head in the 6 degrees of freedom (6 DoF, forward/backward, up/down, left/right, pitch, roll or yaw) as the body relaxes. Even if a patient remains awake there can be movement of the head through muscle relaxation. Normal movement of the head due to breathing can also lower the possible resolution of a PET or MRI scan. Poor image quality can lead to misdiagnosis and/or missed diagnosis.

Movement is also an issue for imaging of other parts of the body. For instance, imaging of the chest region can be degraded by breathing movement and imaging of the heart degraded by cardiac motion.

Attempts have been made to overcome the movement problem by correcting the obtained data for movement. To do this the movement of a patient during a scan must be accurately tracked. Typically the approach taken has been to place markers on the body and to track the markers using a camera and imaging software to track the markers. The technique achieves good result in a research setting but is completely impractical in a clinical setting. The additional time required to attach numerous markers is costly. The various ways of attaching the markers (glue, tape, goggles, caps, helmets) are invasive, uncomfortable, and for many patients, distressing. Furthermore, even if these problems are overlooked, there is risk of the markers independently moving and thus defeating their purpose. There is also the problem that for medical imaging modalities the space for placing markers and tracking equipment is very restricted.

There has recently been proposed one motion tracking system that does not require markers. It is described in a recent journal article [Motion Tracking for Medical Imaging: A Nonvisible Structured Light Tracking Approach; Olesen et. al; IEEE Transaction on Medical Imaging, Vol. 31, No. 1, January 2012]. This article describes a system that illuminates the face of a patient with a pattern of infrared light that is viewed by a CCD camera. The technique relies upon generating a point cloud image of key facial features, particularly the bridge of the nose, and tracking changes due to movement.

The article usefully lists the requirements of a successful movement tracking system in a clinical environment. The requirements are:

1) The registration of the position must be estimated simultaneously so that a detected PET event known as a line of response (LOR) can be repositioned before the PET image reconstruction;
2) The tracking volume must cover the range of the possible head motion in the PET scanner;
3) The system must fit the narrow geometry of the PET scanner;
4) The accuracy of the tracking system has to be better than the spatial resolution of the PET scanner, otherwise the motion correction will increase the blurring instead of reducing it;
5) The system must not interfere with the PET acquisition;
6) The sample frequency has to be at least twice as high as the frequency of head motion to avoid aliasing, according to the Nyquist criterion.

The article goes on to list the clinical requirements for an effective tracking system:

1) Simple to use with a preference for a fully automated system;
2) The tracking system must have an easy interface with the PET scanner;
3) It must be robust and have a flexible design to be a part of the daily routine;
4) The system must be comfortable for the patients, since an uncomfortable patient will introduce motion which is counterproductive for both the patient's well being and the image quality;
5) Finally, the hygiene requirements of hospital use have to be met.

At least one additional requirement has been overlooked; the system must be economically viable.

### SUMMARY OF THE INVENTION

In one form, although it need not be the only or indeed the broadest form, the invention resides in a method of improving resolution in medical imaging of a patient including the steps of:

capturing scanner data of the patient from a medical imaging device;
capturing video image data of the patient;
tracking movement of the patient using tracking algorithms applied to the video image data;
extracting movement correction data from the video image data; and
correcting the scanner data with the movement correction data to produce a medical image of the patient with improved resolution.

The step of extracting movement correction data preferably includes the steps of calibrating the movement correction data against the scanner data to obtain a calibration factor and calibrating the video image data with the calibration factor.

Alternatively, the step of capturing video images of the region may include resolving distance ambiguity by including a fiducial as a reference. The fiducial could be an interpupillary distance of the patient. Alternatively the step of capturing video images may be by a stereo camera.

Preferably the tracking algorithm is a facial recognition algorithm and the medical imaging device produces medical images of the head of the patient.

The video images are suitably captured by a digital camera, such as a webcam.

The movement correct data is suitably calculated and applied across six degrees of freedom. The six degrees of freedom are forward/backward, up/down, lef/right, pitch, roll and yaw.

In another form the invention resides in a movement detection system for use in medical imaging comprising:

a camera;
a signal processor adapted to analyse signals obtained from the camera;
face recognition software running on the signal processor that identifies facial features and tracks movement of the identified features to produce movement correction data; and
an image processor that acquires scanner data from a medical imaging device and corrects the scanner data using the movement correction data.

Further features and advantages of the present invention will become apparent from the following detailed description.

### BRIEF DESCRIPTION OF THE DRAWINGS

To assist in understanding the invention and to enable a person skilled in the art to put the invention into practical effect, preferred embodiments of the invention will be described by way of example only with reference to the accompanying drawings, in which:

FIG. 1 is a sketch of movement correction hardware on an PET scanner;

FIG. 2 demonstrates the movement problem;

FIG. 3 is a block diagram of a movement tracking system;

FIG. 4 depicts a calibration process;

FIG. 5 is a block diagram of a preferred movement tracking system;

FIG. 6 is a plot of a sample patient's head movement in the X, Y and Z axes during a scan;

FIG. 7 are FFT plot of the data in FIG. 4;

FIG. 8 is a plot of movement in Pitch, Yaw and Roll;

FIG. 9 are FFT plot of the data in FIG. 6; and

FIG. 10 demonstrates the improvement in an image.

### DETAILED DESCRIPTION OF THE INVENTION

Embodiments of the present invention reside primarily in a movement correction system for medical imaging. Accordingly, the elements and method steps have been illustrated in concise schematic form in the drawings, showing only those specific details that are necessary for understanding the embodiments of the present invention, but so as not to obscure the disclosure with excessive detail that will be readily apparent to those of ordinary skill in the art having the benefit of the present description.

In this specification, adjectives such as first and second, left and right, and the like may be used solely to distinguish one element or action from another element or action without necessarily requiring or implying any actual such relationship or order. Words such as “comprises” or “includes” are intended to define a non-exclusive inclusion, such that a process, method, article, or apparatus that comprises a list of elements does not include only those elements but may include other elements not expressly listed, including elements that are inherent to such a process, method, article, or apparatus.

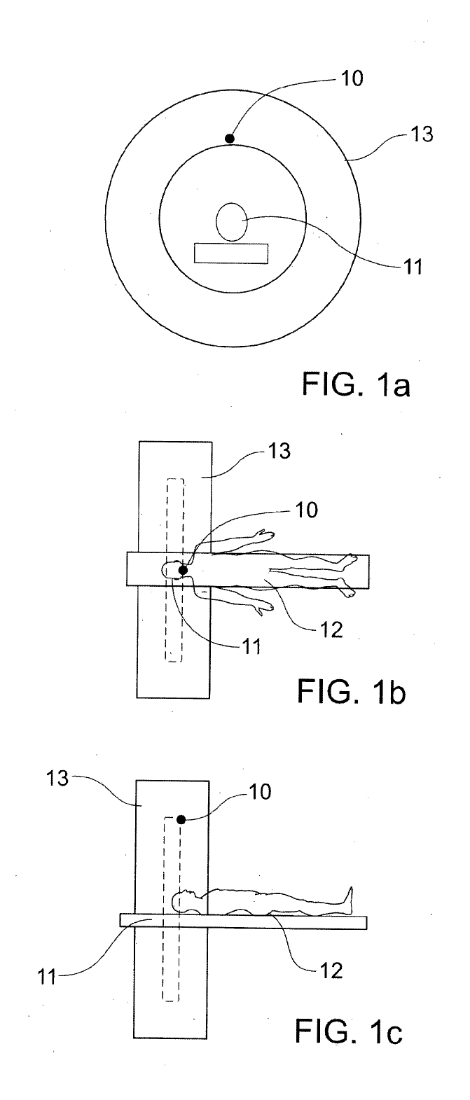

Referring to FIG. 1 there is shown a sketch of a camera 10 positioned to observe the head 11 of a patient 12 during data acquisition in a PET scanner 13. For the purpose of explanation the movement tracking system is described in application to obtaining a PET image, but the invention is readily applicable to any medical image modality, including CT and MRI.

FIG. 1a shows an end view indicating the position of the head 11 of the patient 12 in the scanner 13. The camera 10 is positioned centrally above the patient. FIG. 1 b shows a top view for FIG. 1a and FIG. 1 c shows a side view of FIG. 1a. As can be seen from FIG. 1 b and FIG. 1 c, the camera is positioned to view the patient at a slight angle. The slight angle is due to the camera being position out of the line of the detector crystals of the scanner 13. An alternate approach would be to use a fibre optic positioned directly above the patient. This could be achieved by removing a single detector and replacing it with the tip of a fibre optic. Another option would be to manufacture the camera into the scanner.

The camera 10 may be a commercially available device capable of obtaining a high definition image of a face. The inventors have found that a webcam is adequate for the purposes of demonstration, but recognize that it is probably too bulky for commercial implementation.

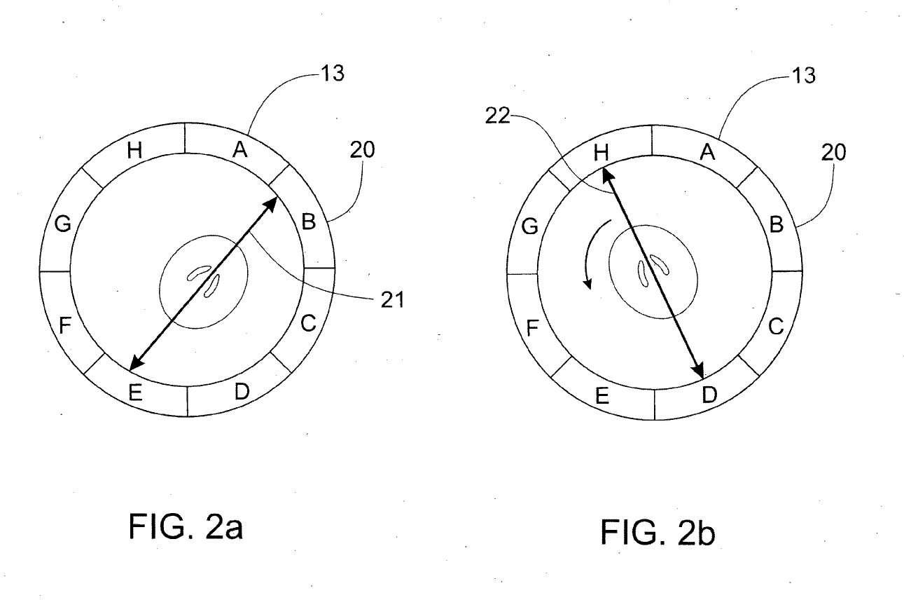

The problem being addressed is made clear in FIG. 2. In FIG. 2a the PET detectors 20 are shown conceptually and labeled as A though H. A real PET scanner has a ring of, for example, 624 crystal detectors with a depth of 52 detectors. If the patient is correctly positioned and still, a PET event generates signals at a pair of detectors, say B and E, and the correct line of response 21 is determined. However, if the patient moves by rolling to the right, as indicated in FIG. 2b, a line of response 22 is assigned to detectors H and D, which is incorrect. The motion is observed by the camera 10 and, as explained below, correction to the raw data is made so that the event is correctly assigned to the direction BE instead of HD.

The video image from the camera 10 is captured using the software supplied with the camera. The image is analysed with any suitable face tracking software. For convenience the inventors have used free software called FaceTrackNoIR which incorporates the FaceAPI tool from Seeing Machines Limited of Canberra, Australia. The movement tracking algorithms generate tracking data that resolves to the 6 degrees of freedom (6 DoF) required to describe a body in space, X, Y, Z, Pitch, Yaw and Roll. For ease of reference the Z axis is taken to be the axis of view of the camera, the X axis is a left or right movement, the Y axis is a neck extension or retraction, Pitch is nodding of the head, Roll is tilting the head left and right, and Yaw is looking left and right.

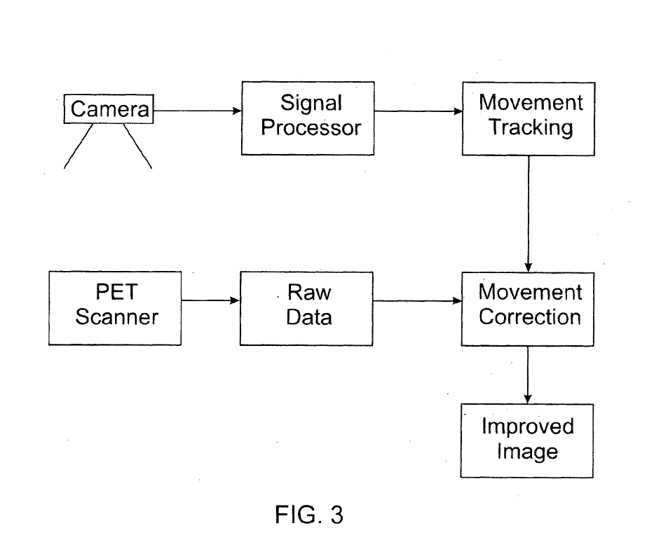

The steps of analysis are set out schematically in FIG. 3. The camera 10 captures an image which is pre-processed by a signal processor, which may also run the movement tracking algorithms to calculate the patients head position in space with respect to the (6 DoF) (or the movement tracking algorithms may be run in a separate processor). Raw data from an imaging device (MRI, CT, PET) is corrected using the movement tracking data to produce an improved image.

If a single camera is used there may be ambiguity in distance measurements as the single camera is unable to determine depth. This can be avoided by using a stereo camera.

Another approach is to apply a scaling factor to the x, y and z plane movements to correct for the object (patient) distance from the camera. This distance may be estimated from the geometry of the imaging device and the location of the camera. For instance, the distance from the camera to the bed of the imaging device is known so the distance to the back of the head of the patient is known. A measurement of the size of the head of the patient can be an input to the analysis algorithms to provide the scaling factor.

The calibration may also be achieved by use of a fiducial. The fiducial may be a ruler or grid of known dimensions that is measured in the image and appropriate scaling applied. The fiducial could also be a known facial measurement, such as the interpupillary distance.

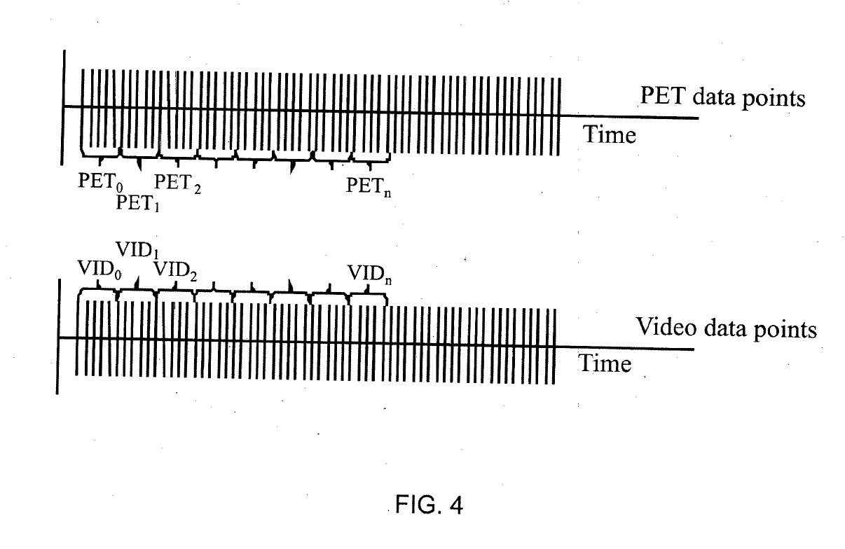

The preferred approach to resolve the distance ambiguity is by calibrating the movement correction data against the scanner data. This process is explained by reference to FIG. 4 using the example of a PET scanner. The PET scanner produces a list file of data against time. The PET image is reconstructed from the data file using reconstruction software provided with the scanner. Typically several million data points are used in image reconstruction. Absolute measurements are inherent in the reconstructed PET data due to the nature of the imaging equipment. Basically, the geometry of the imaging equipment is known and calibrated. Unfortunately a minimum number of data points are needed to reconstruct a PET image and movement of the target can occur within the time needed to acquire this minimum number of data points.

One solution is to average a minimum time block of PET data and calibrate against an equivalent block of video data. The calibration is applied to all video data points and then each individual PET data point (event) within the block is corrected for movement using the corresponding video data point. A suitable time block is 10 seconds.

For each PETₙ block its motion is determined with respect to PET₀. Motion may be determined using known registration techniques such as, but not limited to, mutual information-based methods [Image Registration Techniques: An overview; Medha et. al; International Journal of Signal Processing, Image Processing and Pattern Recognition, Vol. 2, No. 3, September 2009] to align the PETₙ image block with the PET₀ image block. This 6 DoF movement required to align image blocks PETₙ and PET₀ is known as the PET_MOTIONₙ.

For each VIDₙ block its motion is determined with respect to VID₀. Motion may be determined by taking the average motion of each VIDₙ block and calculating it displacement with respect to VID₀.

VID_MOTIONₙ=VIDₙ−VID₀

A calibration value may then be calculated using each PET_MOTION and VID_MOTION block.

Kₙ=PET_MOTIONₙ/VID_MOTIONₙ

The mean of all Kₙ values determine the calibration value that is to be applied to all of the video motion data events.

$$K = \frac{1}{n} \sum {K}_{n}$$

The calibration factor K may be calculated using all of the available blocks or just the minimum required number to provide a statistically accurate value for K. Furthermore, statistical tests may be applied to eliminate some data. For instance, the standard deviation of measurements in a 10 second bin may be used to eliminate blocks of data that have a very high standard deviation. Other statistical tests will be well known to persons skilled in the art.

The correction (K) is applied to all the video data.

VID_{corrected}=K*VID

Motion correction is now applied to the PET data events based on VID_{corrected} to improve resolution at an event level (or more correctly to reduce loss of resolution due to blurring caused by movement).

Although the technique is described in respect of calibration against the first block of PET data, the technique is not limited in this way. Calibration can be performed against any data block or the same process can be followed using a CT scan or MR scan taken immediately before the PET scan.

The calibration process may be applied with any scanner data. It may be summarised as including the steps of: calculating a scanner data correction by registering time-averaged blocks of scanner data to a selected block of scanner data; calculating a video image data correction by registering time-averaged blocks of video image data to a selected block of video image data; calculating a calibration value for each pair of scanner data correction and video image data correction, the pairs of scanner data correction and video image data correction being matched in time; averaging the calibration values to obtain a calibration factor; and applying the calibration factor to the video image data.

In broad terms, as mentioned above, the raw data from the imaging device consists of a list of events with a time stamp for each event. The movement data consists of a time sequence of images from which movement over time is determined. For a particular event the patient position at the time of the event is compared with the initial patient position. If the patient has moved the degree of movement is determined and the line of response 22 is shifted by the determined 6 DoF movement to originate from the correct location. The event is then recorded as having been detected by two different crystals than those that actually recorded the event.

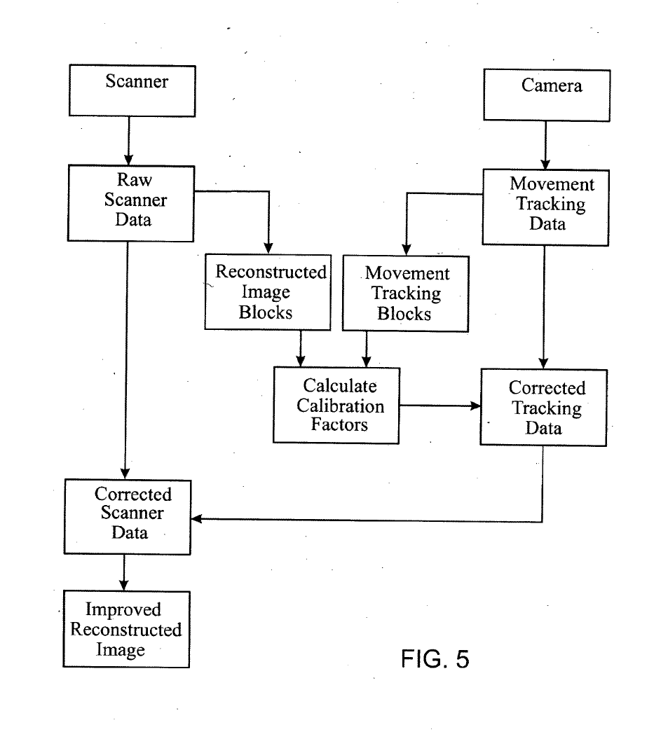

The overall process, using the preferred calibration approach, is depicted in FIG. 5. The scanner (for instance a PET scanner) produces raw scanner data in the form of a list file with a time stamp for each line of data. An image is reconstructed from the minimum block of data that can provide a useful image. The inventors have found that this is 10 seconds for data from a PET scanner. The camera generates video image data that is analysed using movement tracking algorithms to produce blocks of movement tracking data. A calibration factor is calculated and the tracking data is corrected in the manner described above. The corrected tracking data is then used to correct the scanner data to remove the effect of movement of the patient during a scan. The corrected scanner data, in the form of a corrected list file, is then used to produce a reconstructed image by the software provided with the scanner.

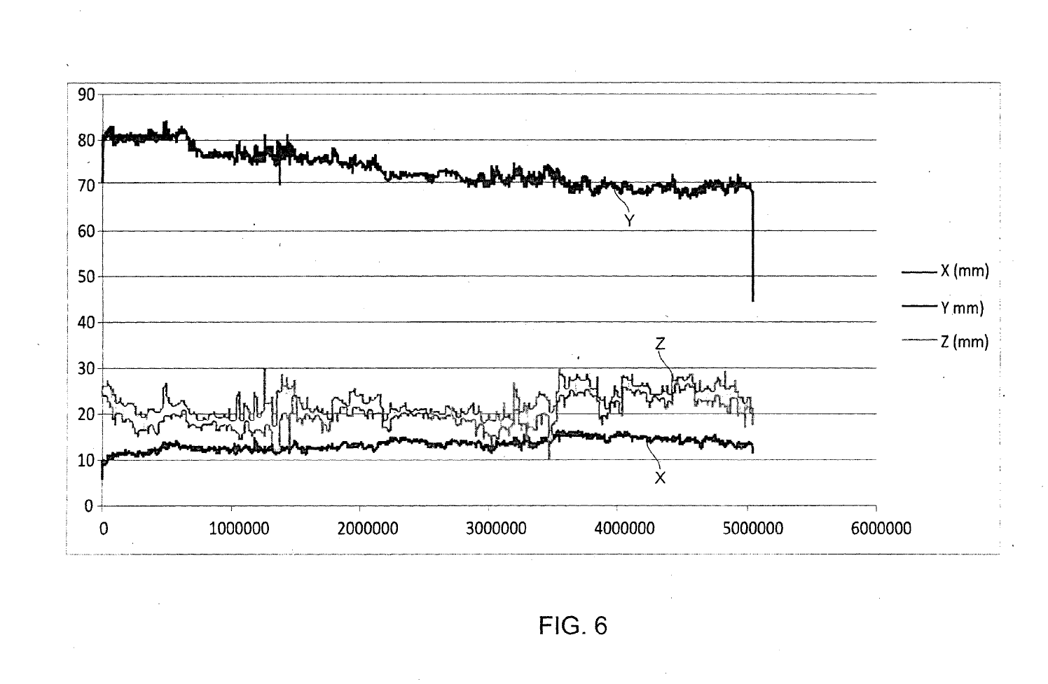

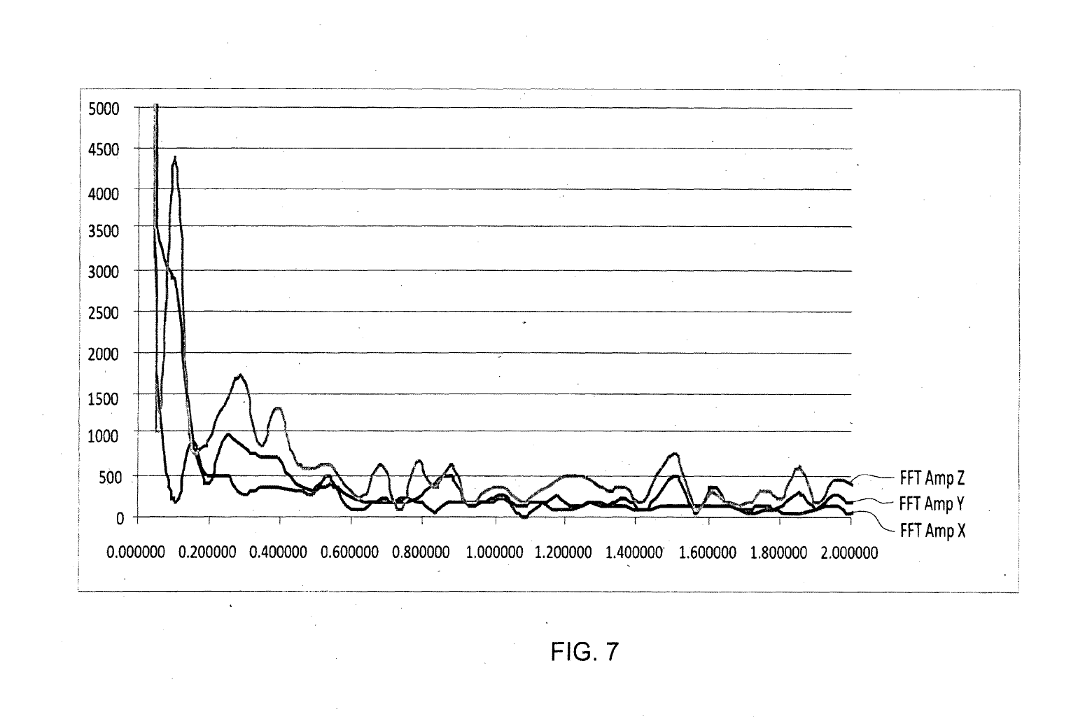

By way of example, FIG. 6 shows X (bottom), Y (top), and Z (middle) movement plots during a PET scan. As can be seen, there is significant drift in the Y position over the duration of the scan and a lot of minor movement in the Z direction. FIG. 7 shows a Fourier transform of the movement data that demonstrates the patterns of movement, for example, a respiratory motion artifact would appear in the Fourier Transform plot as a high amplitude curve centred over a low frequency of about 0.1-0.5 Hertz. These Fourier plots indicate that the patient movements in this case are random and therefore unpredictable. Such FFT of image data from the thorax or abdomen can allow extraction of physiologic data such as respiration and cardiac contraction to facilitate processing of physiologic gated images (for example to show a beating heart image or to freeze movement of a chest lesion).

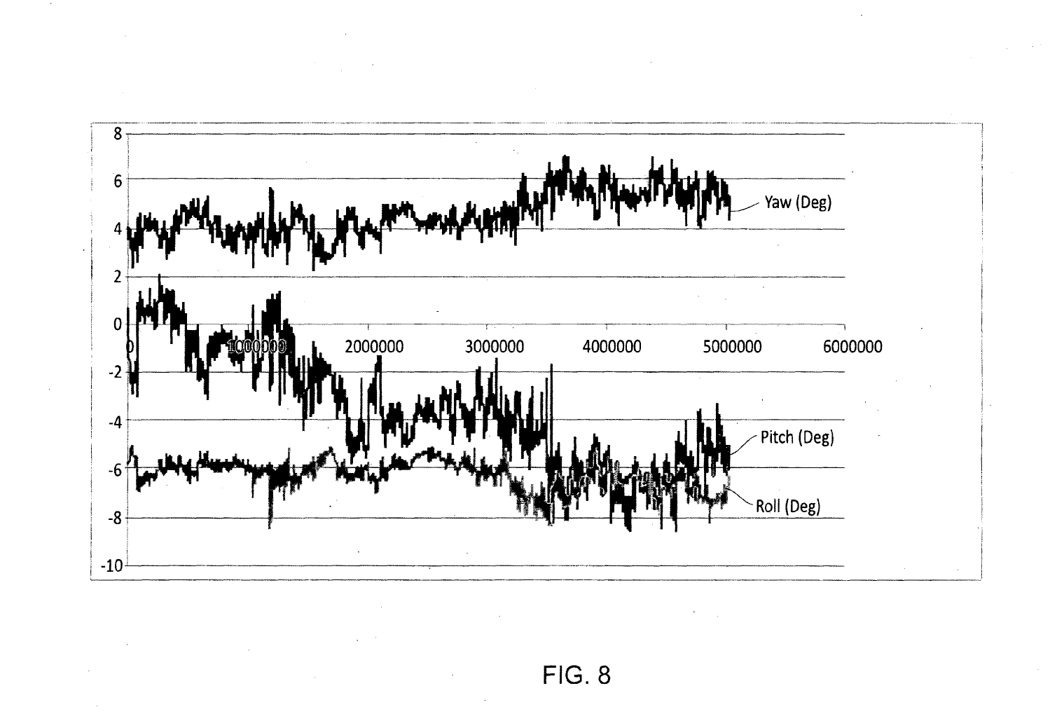

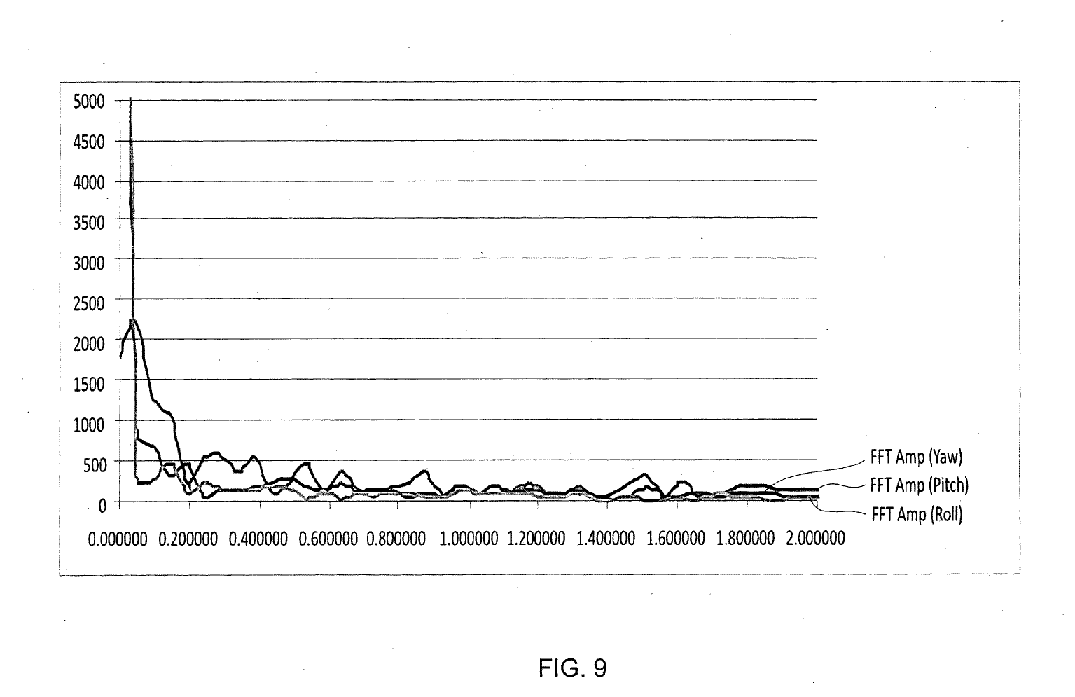

The corresponding plots of Pitch (middle), Yaw (top) and Roll (bottom) are shown in FIG. 8. It is evident that there is a drift in Pitch over the duration of the scan as the patient becomes relaxed and the head rotates towards the body and minor movement in Yaw and Roll. FIG. 9 shows the respective Fourier transform and may also show physiologic data such as respiration and cardiac contraction.

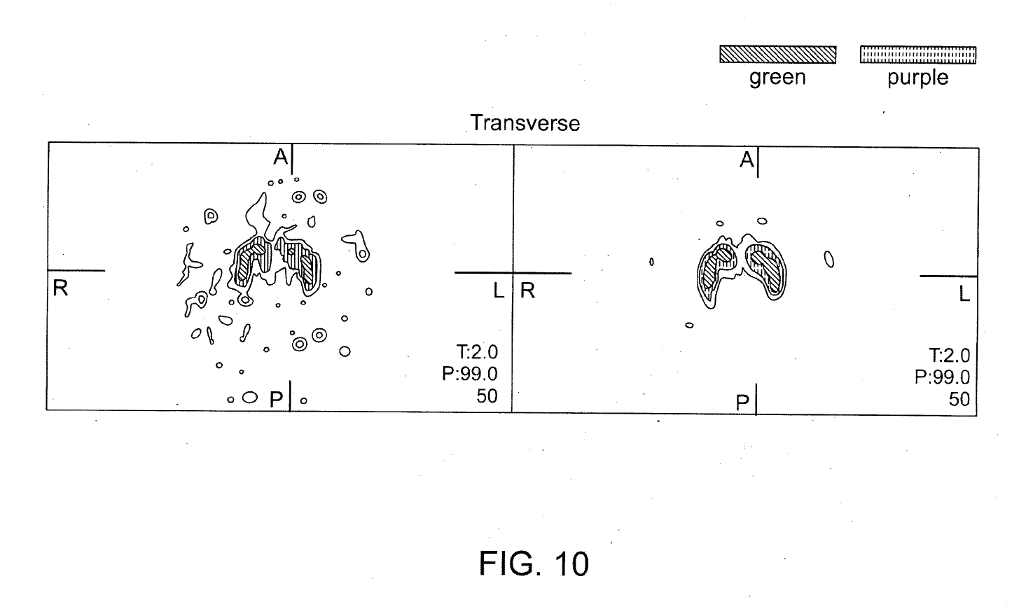

A PET image acquired with the scan represented in FIGS. 6-9 will have a resolution limited by the movement of the patient rather than by the intrinsic resolution of the machine. However, the raw data may be corrected to improve the resolution. This is demonstrated in the images of FIG. 10 which show Flourine-18-FDOPA PET brain images. FDOPA has high uptake in the basal ganglia of the brain (the central areas bilaterally). The initial transverse image (left) shows uptake in the basal ganglia to be more irregular and less intense than uptake in the image (right) which has been corrected for motion. Similarly the scattered blotches in the remainder of the brain and scalp (due to image noise resulting from head movement during acquisition) is markedly reduced on the motion corrected image.

The above description of various embodiments of the present invention is provided for purposes of description to one of ordinary skill in the related art. It is not intended to be exhaustive or to limit the invention to a single disclosed embodiment. As mentioned above, numerous alternatives and variations to the present invention will be apparent to those skilled in the art of the above teaching. Accordingly, while some alternative embodiments have been discussed specifically, other embodiments will be apparent or relatively easily developed by those of ordinary skill in the art. Accordingly, this invention is intended to embrace all alternatives, modifications and variations of the present invention that have been discussed herein, and other embodiments that fall within the spirit and scope of the above described invention.

1. A method of improving resolution in medical imaging of a patient including the steps of:
capturing scanner data of the patient from a medical imaging device;
capturing video image data of the patient with a camera positioned in or on the medical imaging device centrally above the patient;
tracking movement of the patient using tracking algorithms applied to the video image data;
extracting movement correction data from the video image data; and
correcting the scanner data with the movement correction data to produce a medical image of the patient with improved resolution.

2. The method of claim 1 wherein the step of extracting movement correction data includes the steps of calibrating the movement correction data against the scanner data to obtain a calibration factor and calibrating the video image data with the calibration factor.

3. The method of claim 2 wherein calibration of the movement correction data includes the steps of:
calculating a scanner data correction by registering time-averaged blocks of scanner data to a selected block of scanner data;
calculating a video image data correction by registering time-averaged blocks of video image data to a selected block of video image data;
calculating a calibration value for each pair of scanner data correction and video image data correction, the pairs of scanner data correction and video image data correction being matched in time;
averaging the calibration values to obtain a calibration factor; and
applying the calibration factor to the video image data.

4. The method of claim 3 wherein the blocks of scanner data and the blocks of video image data are ten second blocks.

5. The method of claim 3 wherein the selected block of scanner data is the first block of scanner data and the selected block of video image data is the first block of video image data.

6. The method of claim 1 wherein the tracking algorithms are facial recognition algorithms.

7. The method of claim 6 wherein the medical imaging device generates images of a head of the patient.

8. The method of claim 1 wherein the video images are captured by a digital camera.

9. The method of claim 1 wherein the step of capturing video image data of the patient includes resolving distance ambiguity by including a fiducial as a reference.

10. The method of claim 1 wherein the movement correction data is calculated and applied across six degrees of freedom.

11. A movement detection system for use in medical imaging comprising:
a camera positioned in or on a medial imaging device centrally above the patient;
a signal processor adapted to analyse signals obtained from the camera;
face recognition software running on the signal processor that identifies facial features and tracks movement of the identified features to produce movement correction data; and
an image processor that acquires scanner data from a medical imaging device and corrects the scanner data using the movement correction data.

12. The movement detection system of claim 11 wherein the camera is a stereo camera.

13. The movement detection system of claim 11 wherein the medical imaging device is selected from a PET scanner, CT scanner or MR scanner.

14. The movement detection system of claim 11 further comprising means for calibrating the movement correction data against the scanner data.

15. The movement detection system of claim 11 further comprising a fiducial for calibration of the movement correction data.
